(19) United States
(12) Patent Application Publication
(10) Pub. No.: US 2010/0035845 A1
(43) Pub. Date: Feb. 11, 2010

(54) TIGECYCLINE FORMULATIONS

(75) Inventors: Christian Luther OFSLAGER, Newburg, NY (US); Nataliya BAZHINA, Tappan, NY (US); Mahdi Bakir FAWZI, Morristown, NJ (US); Syed Muzafar SHAH, East Hanover, NJ (US); Gurmukh Das CHANANA, West Nyack, NY (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/535,957

(22) Filed: Aug. 5, 2009

Related U.S. Application Data
(60) Provisional application No. 61/086,552, filed on Aug. 6, 2008.

(51) Int. Cl. A61K 31/65

(52) U.S. Cl. 514/152

(57) ABSTRACT

The invention is directed to a frozen pharmaceutical formulation suitable for administration to a subject parenterally, comprising a therapeutically effective amount of tigecycline and an agent selected from the group consisting of lactose, dextrose, glucose, mannose, sucrose, ribose, xylose and a combination thereof, wherein the formulation in a pre-frozen state at about 22° C. or in an unfrozen state at about 22° C. has a pH in the range of from 4.0 to 5.5. Preferably, the formulation is suitable for storage at or below about −20° C. over a period of at least about 2 months, preferably 6 months, more preferably 26 months. Alternatively, the formulation is suitable for storage at about 22° C. over a period of about 24 hours.

This application claims priority from copending U.S. Provisional Application No. 61/086,552, filed Aug. 6, 2008, the entire disclosure of which is hereby incorporated by reference.

### FIELD

This invention relates to pharmaceutical formulations of tigecycline, and more particularly to pharmaceutical formulations of tigecycline suitable for storage at or below about −20° C. The formulations are suitable for intravenous administration and have a viable shelf life.

### BACKGROUND OF THE INVENTION

Tigecycline is a glycylcycline antibiotic, i.e., a t-butylglycyl substituted naphthacenecarboxamide free base, and an analog of the semisynthetic tetracycline, minocycline. Tigecycline is marketed under the tradename TYGACIL® in lyophilized form in a vial.

U.S. Pat. Nos. RE40,183 and RE40,086 disclose and claim, among other subject matter, a genus that encompasses tigecycline, salts thereof, compositions thereof, and methods of treatment of certain indications. Other inventions relating to tigecycline include U.S. Patent Publication Nos.: 2006-0247181, titled “Tigecycline compositions and methods of preparation”; U.S. Patent Publication Nos.: 2007-0259439, titled “Determination of antibiotic concentration in bone”; 2007-0026080, titled “Manufacturing process for tigecycline”; 2006-0094668, titled “Co-administration of tigecycline and digoxin”; and 2005-0130252, “Stabilized susceptibility tests of aerobic pathogens”. All of the above patents and patent publications are incorporated by reference herein in their entirety.

Tigecycline is supplied in the lyophilized form and therefore must be reconstituted prior to intravenous administration. Simple oral immediate release prototypes containing tigecycline have resulted in poor bioavailability in animals. (Petersen et al., Antimicrobial Agents and Chemotherapy, April 1999, Vol. 43, No. 4 p. 738-744.). The reconstitution required by a lyophilized vial product is a skilled pharmaceutical procedure that must be performed using aseptic techniques to ensure stability of the reconstituted and admixed product. In addition, a reconstituted product may have a relatively short refrigerated shelf life compared to products prepared according to other techniques. And such components required to prepare the reconstituted solution as vials, needles and bags must be properly discarded. In short, it is desirable to provide an alternative drug product design that may reduce the potential for pharmacist errors, such as are described in, for example, Journal of Nursing Law, 2005, 10, 201-207.

Alternative drug product designs include storing the product in a frozen bag that can be kept in a freezer for long periods of time before thawing at the time of the intended use. However, lowering the temperature does not necessarily increase the stability of a composition. For example, Am. J. Health Syst. Pharm., 2004, 61, 38-45 states that samples of ertapenem solutions frozen at −20° C. showed extreme variability in stability, and J. Clin, Pharm. and Therapeutics (1989) 14, 45-52 reports that freezing solutions of amoxycillin sodium in normal saline or in glucose markedly reduced stability. Hospital Pharmacy, 1976, Vol. 11, 178-79, does report that a number of antibiotics (cephalothin, penicillin, and carbenicillin) retained essentially all of their activity after 14-23 days of frozen storage, but that clindamycin lost about 13% of its label activity after 23 days. Therefore, lowering the temperature has an unpredictable effect on the stability of a composition, and may, in fact, actually decrease its stability depending on the component.

Accordingly, there remains a need to develop alternative compositions containing tigecycline for the further benefit of patients.

### SUMMARY OF THE INVENTION

This invention relates to pharmaceutical formulations of tigecycline suitable for storage at or below about −20° C. The formulations are suitable for intravenous administration and have a viable shelf life. The formulations of the present invention are advantageous over the lyophilized product as they are premixed and stable for periods longer than the commercially available vial product at refrigerated temperature. Additionally, the formulations of the present invention may be stored in a prepared bag for use by medical personnel, further reducing any potential for problems such as dosage calculation errors and discarding needle sticks.

The invention is directed to a frozen pharmaceutical formulation suitable for administration to a subject parenterally, comprising a therapeutically effective amount of tigecycline and an agent selected from the group consisting of lactose, dextrose, glucose, mannose, sucrose, ribose, xylose and a combination thereof, wherein the formulation in a pre-frozen state at about 22° C. or in an unfrozen state at about 22° C. has a pH in the range of from 4.0 to 5.5.

The invention is also directed to a pharmaceutical formulation suitable for administration to a subject parenterally, comprising a therapeutically effective amount of tigecycline and an agent selected from the group consisting of lactose, dextrose, glucose, mannose, sucrose, ribose, xylose and a combination thereof, wherein the formulation has a pH in the range of from 4.0 to 5.5.

Preferably, the formulation is suitable for storage at or below about −20° C. over a period of at least about 2 months, preferably 6 months, more preferably 26 months. Alternatively, the formulation is suitable for storage at about 22° C. over a period of about 24 hours.

The invention is also directed to a process of making a frozen pharmaceutical formulation, comprising the steps of:
a. dissolving tigecycline and an agent selected from the group consisting of lactose, dextrose, glucose, mannose, sucrose, ribose, xylose and a combination thereof into a suitable liquid, forming a premixed solution;
b. adjusting the pH of the premixed solution to a range of 4.0-5.5;
c. filling one or more containers with the premixed solution; and
d. storing the one or more containers of premixed solution at a temperature below the freezing point of the liquid.

The invention is also directed to a pharmaceutical formulation made by the above process.

The invention is also directed to a process of making a frozen pharmaceutical formulation, comprising the steps of:
a. dissolving tigecycline and agent selected from the group consisting of lactose, dextrose, glucose, mannose, sucrose, ribose, xylose and a combination thereof into a suitable liquid, forming a premixed solution;
b. filling one or more containers with the premixed solution;
c. storing the one or more containers of premixed solution at a temperature below the freezing point of the liquid; and
d. adding an acidifying agent to form a mixture wherein the amount of acidifying agent is such that the pH of the resulting mixture when measured at a temperature of about 22° C. is 4.0-5.5.

The invention is also directed to a pharmaceutical formulation made by the above process.

The invention is also directed to a kit comprising (a) a flexible bag, and (b) a frozen pharmaceutical formulation as described herein, wherein the frozen pharmaceutical formulation is contained in the bag, which may be, for example, a plastic bag.

The invention is also directed to a kit comprising (a) a vial, and (b) a frozen pharmaceutical formulation as described herein, wherein the frozen pharmaceutical formulation is contained in the vial.

### DEFINITIONS

As used herein, “tigecycline epimer” is the epimer of tigecycline at the epimerizable carbon bearing a dimethylamino group.

As used herein, a formulation “suitable for storage” is a formulation that does not form more than 4.5% wt/wt of total impurity content in a given time period. “Total impurity content” is the total of the amount of all the impurities in the tigecycline formulation, including the epimer of tigecycline, as a percentage of the initial weight of tigecycline.

As used herein, a “therapeutically effective amount” of tigecycline is an amount effective to treat a condition in a mammal related to bacterial infections. Such infections include infections related to tetracycline resistant strains infections related to strains which are normally susceptible to tetracyclines. These include strains of E. coli, S. aureus and E. faecalis, containing the tetM resistance determinants (such as S. aureus UBMS 88-5, S. aureus UBMS 90-1 and 90-2, E. coli UBMS 89-1 and 90-4). Such bacterial infections also include complicated abdominal infections, complicated skin structure infections, osteomyelitis, diabetic foot, hospital acquired pneumonia, community acquired pneumonia, ventilator acquired pneumonia.

### DESCRIPTION OF THE DRAWINGS

FIG. 1 shows the effect of pH on the total impurity content without dissolved oxygen control at 25° C.

### DETAILED DESCRIPTION OF THE INVENTION

The invention is directed to a frozen pharmaceutical formulation suitable for administration to a subject parenterally, comprising a therapeutically effective amount of tigecycline and an agent selected from the group consisting of lactose, dextrose, glucose, mannose, sucrose, ribose, xylose and a combination thereof, wherein the formulation in a pre-frozen state at about 22° C. or in an unfrozen state at about 22° C. has a pH in the range of from 4.0 to 5.5.

Preferably, the formulation is suitable for storage at or below about −20° C. over a period of at least about 26 months. Alternatively, the formulation is suitable for storage at about 22° C. over a period of about 24 hours. In one embodiment, the agent is selected from the group consisting of lactose, dextrose, glucose, mannose, and a combination thereof.

In another embodiment, the agent is lactose in a concentration within the range of about 40 mg/ml to about 80 mg/ml.

In one embodiment of the formulation of the invention, the concentration of tigecycline epimer in the formulation is at or below about 3%, preferably at or below about 2%, of the concentration of tigecycline.

In one embodiment of the formulation of the invention, the effective amount of tigecycline is about 150 mg. The concentration of tigecycline may be, as an example, within the range of about 0.1 mg/ml to about 2.0 mg/ml, preferably from about 0.5 to about 1.5 mg/ml.

In another embodiment of the invention, the pH may be from 4.5 to 5.1, for example from 4.8 to 5.1.

In another embodiment of the invention, the formulation contains at least about 98.0% tigecycline as a percentage of the initial weight of tigecycline in the composition after up to about 42 days at a temperature of about −20° C.

In another embodiment of the invention, the formulation contains at least about 90.0% tigecycline as a percentage of the initial weight of tigecycline in the composition after up to 36 months, such as for example up to 6 months, at a temperature of about −20° C. or lower than −20° C. The temperature may be of about −20° C. to about −70° C.

As another example of the embodiment of the invention, the agent selected from the group consisting of an agent selected from the group consisting of lactose, dextrose, glucose, mannose, sucrose, ribose, xylose and a combination thereof is lactose, dextrose, or a combination thereof.

As used herein, “lactose” and “dextrose” encompass both hydrous and anhydrous forms of lactose and dextrose. Lactose may be, for example, in hydrous form or in anhydrous form. For example, lactose may be in the form of lactose monohydrate. Lactose may be present in a concentration within the range of about 40 mg/ml to about 80 mg/ml. Dextrose may be, for example, in the hydrous form or in the anhydrous form. Dextrose may be present in a concentration within the range of about 40 mg/ml to about 80 mg/ml.

The stability of the formulations allows the present invention to be suitable for storage at or below about −20° C., for at least about 26 months. Before use the frozen formulation is thawed and remains viable for about 24 hours at room temperature or at a temperature of about 22° C.

The invention is also directed to a process of making a pharmaceutical formulation, comprising the steps of:
a. dissolving tigecycline and an agent selected from the group consisting of an agent selected from the group consisting of lactose, dextrose, glucose, mannose, sucrose, ribose, xylose and a combination thereof into a suitable liquid, forming a premixed solution;
b. adjusting the pH of the premixed solution to a range of 4.0-5.5;
c. filling one or more containers with the premixed solution; and
d. storing the one or more containers of premixed solution at a temperature below the freezing point of the liquid.
   The invention is also directed to a pharmaceutical formulation made by the above process.

In one embodiment of the process, the pH of the premixed solution is from 4.0 to 5.1, for example from 4.8 to 5.1.

In one embodiment of the above process the agent selected from the group consisting of an agent selected from the group consisting of lactose, dextrose, glucose, mannose, sucrose, ribose, xylose and a combination thereof is lactose, dextrose or a combination thereof. For example, the agent may be lactose, which is preferably present in a concentration as disclosed herein. In one aspect of this embodiment, tigeycline is dissolved first, followed by lactose. In another aspect of this embodiment, lactose is dissolved first, followed by tigeycline. The process may further comprise the step of adding an amount of dextrose. The dextrose may be added, for example, between step a. and step b., or between step b. and step c. After completion of the addition of dextrose, dextrose is in a concentration as disclosed herein.

In another embodiment of the process, the suitable liquid in step a. is water. In an example of this embodiment, the temperature below the freezing point of the liquid is about −20° C. In an example of this embodiment, the temperature below the freezing point of the liquid is about −20° C. to about −70° C.

The invention is also directed to a process of making a pharmaceutical formulation, comprising the steps of:
a. dissolving tigecycline and an agent selected from the group consisting of lactose, dextrose, glucose, mannose, sucrose, ribose, xylose and a combination thereof into a suitable liquid, forming a premixed solution;
b. filling one or more containers with the premixed solution;
c. storing the one or more containers of premixed solution at a temperature below the freezing point of the liquid; and
d. adding an acidifying agent to form a mixture wherein the amount of acidifying agent is such that the pH of the resulting mixture when measured at a temperature of about 22° C. is 4.0-5.5.

The invention is also directed to a pharmaceutical formulation made by the above process.

In one embodiment of the above process the agent selected from the group consisting of an agent selected from the group consisting of lactose, dextrose, glucose, mannose, sucrose, ribose, xylose and a combination thereof is lactose, dextrose or a combination thereof. For example, the agent may be lactose, which is preferably present in a concentration as disclosed herein. In one aspect of this embodiment, tigeycline is dissolved first, followed by lactose. In another aspect of this embodiment, lactose is dissolved first, followed by tigeycline. The process may further comprise the step of adding an amount of dextrose. The dextrose may be added, for example, between step a. and step b. After completion of the addition of dextrose, dextrose is in a concentration as disclosed herein.

In another embodiment of the process, the suitable liquid in step a. is water. In an example of this embodiment, the temperature below the freezing point of the liquid is about −20° C. In an example of this embodiment, the temperature below the freezing point of the liquid is about −20° C. to about −70° C.

One advantage of the present invention is that the formulation demonstrates long-term stability and enhanced shelf life when prepared at a suitable pH range as disclosed herein. The stability of the formulation is achieved by maintain the formulation within the pH range, which may be obtained by acidification with an acidifying agent such as, for example, hydrochloric acid. The hydrochloric acid is at a suitable concentration, such as from about 0.01 M to about 2 M. Another advantage is that the formulation of the present invention is premixed so it is ready for immediate use upon thawing.

It is important for the stability of the formulation that the pH be maintained within a suitable range. It has been found that a pH may be from 4.0 to 5.5, for example from 4.5 to 5.1. Hydrochloric acid or other suitable acid can be used to adjust the pH downward, and sodium hydroxide, or other suitable base, can be used to adjust the pH upward as needed.

In the process of the invention, after mixing is completed, the premixed solution of tigecycline and lactose is placed into one or more dosage containers. The one or more containers are then stored in a freezer at about −20° C. or lower. Studies have shown that the formulations of the present invention remain viable for at least about 26 months while frozen.

Before use the frozen containers should be thawed in a conventional manner. The formulations will remain viable at room temperature or at about 22° C. for about 24 hours after removal from the freezer.

Under careful study, the stability of different formulations during long-term frozen and short-term thawed storage was assessed. Various formulations were evaluated to ascertain which combinations of components had long-term stability. Parameters assessed included drug concentration, impurities, solution pH, solution color, visual appearance, and particulate matter.

Various solution pHs were evaluated as well. Preferred formulations were stored for up to 26 months frozen.

The advantageousness of the invention can be shown by the following examples, which are meant to be purely illustrative and are not intended to be limiting of the invention in any way. Numerous other examples can readily be envisioned in light of the guiding principles and teachings contained herein.

### Comparative Example 1

The effect of pH on the stability of tigecycline formulations having a concentration of 0.50 mg tigecycline/ml in a Normal Saline solution was studied by analyzing the variation of tigecycline content, total impurity content, and the concentration of tigecycline epimer at different pH values, either in the presence of air or under an atmosphere having an oxygen content of less than 1 ppm, in a flask at 25° C. The results are shown in Tables 1-6.

TABLE 1
Tigecycline content* remaining over time at various pH values
Percentage of tigecycline remaining over time at different pH values
(oxygen content <1 ppm, 25° C.)

| | pH | | | | |
|---|---|---|---|---|---|
| T (hours) | 3.0 | 3.5 | 4.5 | 6.5 | 8.0 |
| 0 | 100.0 | 100.0 | 100.0 | 100.0 | 100.0 |
| 8 | 99.55 | 100.19 | 99.69 | 100.56 | 100.81 |
| 24 | 98.08 | 99.26 | 99.72 | 99.59 | 99.64 |
| 72 | 92.24 | 96.03 | 98.50 | 98.03 | 95.98 |

*±2%

TABLE 2
Tigecycline content* remaining over time at various pH values
Percentage of tigecycline remaining over time at different pH values
(in the presence of air, 25° C.)

| | pH | | | | | | | |
|---|---|---|---|---|---|---|---|---|
| T | 3.0 | 3.5 | 4.0 | 4.5 | 5.0 | 5.5 | 6.5 | 8.0 |
| 0 | 100 | 100 | 100 | 100 | 100 | 100 | 100 | 100 |
| 8 | 100 | 100 | N/A | 100 | N/A | N/A | 100 | 100 |
| 24 | 98 | 100 | 100 | 99 | 101 | 99 | 100 | 95 |
| 72 | 93 | 95 | 98 | 97 | 99 | 97 | 86 | 57 |

*±2%

TABLE 3
Impurity content over time at various pH values
Total impurity content as a % of initial weight of
tigecycline in formulation over time at
different pH values (oxygen content <1 ppm, 25° C.)

Hours

| Ph | 0 | 8 | 24 | 72 |
|---|---|---|---|---|
| 3 | 0.258 | 0.628 | 2.391 | 7.853 |
| 3.5 | 0.245 | 0.559 | 1.478 | 4.913 |
| 4.5 | 0.232 | 0.368 | 0.682 | 2.365 |
| 6.5 | 0.224 | 0.399 | 0.812 | 2.654 |
| 8.0 | 0.22 | 0.443 | 1.214 | 3.461 |

TABLE 4
Impurity content over time at various pH values
Total impurity content as a % of initial weight of
tigecycline in formulation over time
at different pH values (in the presence of air**, 25° C.)

Hours

| pH | 0 | 8 | 24 | 72 |
|---|---|---|---|---|
| 3 | 0.36 | 0.907 | 2.564 | 8.3 |
| 3.5 | 0.35 | 0.737 | 1.727 | 5.521 |
| 4.0 | 0.32 | NA | 1.06 | 3.129 |
| 4.5 | 0.30 | 0.483 | 0.75 | 2.625 |
| 5.0 | 0.29 | NA | 0.691 | 2.632 |
| 5.5 | 0.31 | NA | 0.865 | 3.221 |
| 6.5 | 0.31 | 0.602 | 1.429 | 11.78 |
| 8.0 | 0.30 | 0.924 | 3.737 | 29.654 |

TABLE 5
Content of tigecycline epimer over time at various pH values
Total tigecycline epimer content as a % of initial weight of
tigecycline in formulation over time at different pH values
(oxygen content <1 ppm, 25° C.)

Hours

| pH | 0 | 8 | 24 | 72 |
|---|---|---|---|---|
| 3 | 0.152 | 0.485 | 2.267 | 7.651 |
| 3.5 | 0.133 | 0.383 | 1.351 | 4.671 |
| 4.5 | 0.116 | 0.233 | 0.547 | 1.934 |
| 6.5 | 0.111 | 0.188 | 0.45 | 1.532 |
| 8.0 | 0.107 | 0.192 | 0.425 | 1.516 |

TABLE 6
Content of tigecycline epimer over time at various pH values
Total tigecycline epimer content as a % of initial weight of
tigecycline in formulation over time at different pH values
(in the presence of air**, 25° C.)

Hours

| pH | 0 | 8 | 24 | 72 |
|---|---|---|---|---|
| 3 | 0.19 | 0.83 | 2.27 | 7.58 |
| 3.5 | 0.18 | 0.47 | 1.45 | 4.83 |
| 4.0 | 0.189 | Not tested | 0.90 | 2.90 |
| 4.5 | 0.16 | 0.30 | 0.65 | 1.95 |
| 5.0 | 0.167 | Not tested | 0.49 | 1.69 |
| 5.5 | 0.162 | Not tested | 0.52 | 1.31 |
| 6.5 | 0.15 | 0.26 | 0.43 | 1.08 |
| 8.0 | 0.14 | 0.27 | 0.43 | 1.23 |

**For pH 4.0, pH 5.0, pH 5.5 solutions, no measurements were performed at 8 hours timepoint.

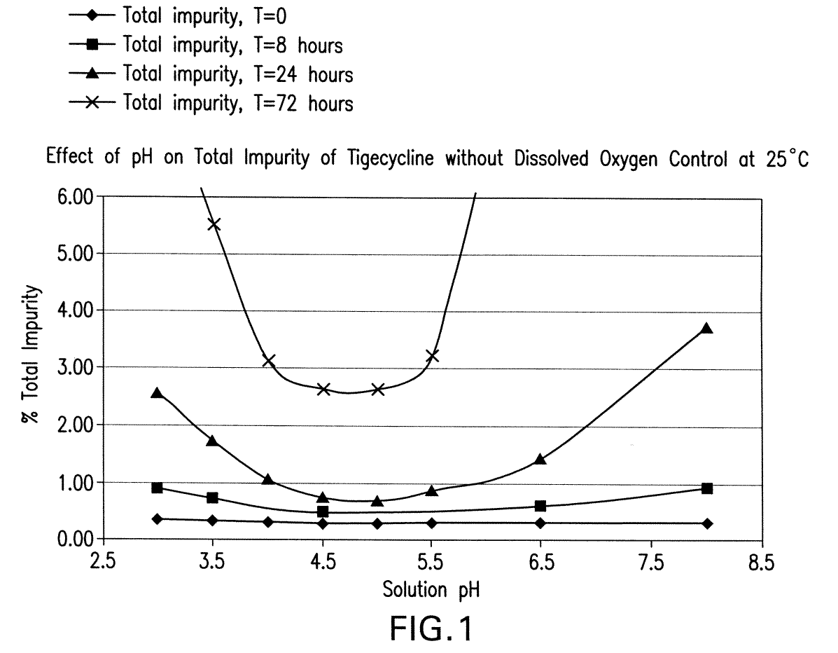

The above results may be also visualized in plot form. As an example, the results shown in Table 4 are depicted in FIG. 1. The plots shown in FIG. 1 result from curve fitting to the individual points.

As shown in Tables 2 and 4, in the presence of air, the pH at which the solution is most stable, based on the total impurity content (Table 4) and the tigecycline content (Table 2) ranges from 4.0 to 5.5, more particularly from 4.5 to 5.0. In an atmosphere where the amount of oxygen is <1 ppm, the pH at which the solution is most stable is 4.5-6.0. At a pH of 4.5-5.0, which is optimal in the presence of air, the total tigecycline content of the formulation is similar to the total tigecycline content when the oxygen level is <1 ppm (Tables 1 and 3). In other words, in this pH range the impact of the oxygen level is small. With regard to the epimer of tigecycline, as shown in Tables 5 and 6, the solution containing the smallest amount of epimer as a function of pH is the solution at pH ranging from 5.5 to 8.0 under inherent conditions, or from 6.5 to 8.0 when the amount of oxygen is <1 ppm.

The foregoing results are unexpected in that the pH range at which the solution contains the least amount of total impurity content and the greatest amount of tigecycline content is not the same, but is instead significantly lower—from 4.0 to 5.5 or from 4.5 to 6.0, depending on whether air is present or whether the amount of oxygen is <1 ppm—than the pH range taught by U.S. Pat. No. 6,207,661 assigned to Baxter, which teaches a pH range of 6.1 to 6.9, preferably 6.3 to 6.7, and which emphasizes the potential disadvantage of a pH lower than 6.5. The pH range at which the solution contains the least amount of total impurity content and the greatest amount of tigecycline content is also significantly lower than the pH range at which the solution of the invention contains the least amount of tigecycline epimer (5.5 to 8.0 in the presence of air, or from about 6.5 to 8.0 when the amount of oxygen is <1 ppm).

All of the above results pertain to measurements at about 25° C. The effect of lowering the temperature below freezing while also varying the pH on the stability of the formulations is discussed further below.

### Comparative Example 2

The stability of formulations obtained from tigecycline in lyophilized form by dissolving it in a Normal Saline solution at pH 6.0 (a) in the absence of lactose and (b) in the presence of lactose was compared over a four-week period, in each case after storing at about −20° C. The results are shown in Tables 7-8.

TABLE 7
Percentage of impurities formed in a Normal Saline solution of 500 mg of tigecycline in 1000 ml of solution at pH 6.0 after
storing at about −20° C. over a two-week period. The pH was adjusted with 1N HCl. The impurity at RRT 0.74 corresponds to the
tigecycline epimer.

| | | Percent Impurities Formed | | | | | | | | | |
|---|---|---|---|---|---|---|---|---|---|---|---|
| Sample | Strength | RRT | RRT | RRT | RRT | RRT | | RRT | RRT | RRT | Total |
| Name | mg/ml | 0.21 | 0.25 | 0.35 | 0.50 | 0.60 | Epimer | 0.78 | 1.06 | 1.64 | Impurities Formed |
| Acceptance | NA | ≦0.2 | ≦0.2 | ≦0.15 | ≦0.2 | ≦0.7 | ≦3.0 | ≦0.2 | ≦0.2 | FIO | ≦6.0 |
| criteria, % | | | | | | | | | | | |
| Initial | 0.52 | ND | ND | ND | ND | 0.01 | 0.14 | ND | ND | 0.10 | 0.25 |
| 2 weeks | 0.53 | ND | ND | ND | 0.05 | 0.28 | 0.25 | ND | ND | 0.11 | 0.69 |
| 4 | 0.40 | 0.20 | 0.04 | 0.62 | 0.33 | 2.49 | 0.14 | 0.08 | 0.47 | 0.26 | 4.63 |
| weeks* | | | | | | | | | | | |

RRT—Relative Retention Time
ND—Not Detected
FIO—For Information only
NA—Not Applicable
*After 4 weeks the tigecycline frozen bags turned green color

TABLE 8
Percentage of impurities formed in a Normal Saline solution of 500 mg of tigecycline and 1000 mg of lactose in 1000 ml of
solution at pH 6.0 after storing at about −20° C. over a two-week period. The pH was adjusted with 1N HCL.

| | | Percent Impurities Formed | | | | | | | | | | | Total |
|---|---|---|---|---|---|---|---|---|---|---|---|---|---|
| Sample | Strength | RRT | RRT | RRT | RRT | RRT | RRT | RRT | RRT | | RRT | RRT | Impurities |
| Name | mg/ml | 0.21 | 0.25 | 0.35 | 0.37 | 0.40 | 0.50 | 0.60 | 0.70 | Epimer | 1.06 | 1.64 | Formed |
| Accep- | NA | ≦0.2 | ≦0.2 | ≦0.15 | ≦0.2 | ≦0.2 | ≦0.2 | ≦0.7 | ≦0.2 | ≦3.0 | ≦0.2 | FIO | ≦6.0 |
| tance | | | | | | | | | | | | | |
| criteria, % | | | | | | | | | | | | | |
| Initial | 0.52 | ND | ND | ND | ND | ND | ND | ND | ND | 0.14 | ND | 0.08 | 0.22 |
| 2 weeks | 0.51 | ND | ND | ND | ND | ND | 0.02 | 0.11 | ND | 0.23 | ND | 0.10 | 0.46 |
| 4 | 0.47 | 0.23 | 0.04 | 0.61 | 0.05 | 0.02 | 0.35 | 2.71 | 0.04 | 0.14 | 0.47 | 0.26 | 4.92 |
| weeks* | | | | | | | | | | | | | |

RRT—Relative Retention Time
ND—Not Detected
FIO—For Information only
NA—Not Applicable
*After 4 weeks the tigecycline frozen bags turned green color

In the relatively short period of two weeks, a significant increase in stability of the formulation containing lactose (Table 8) relative to the formulation that does not contain lactose (Table 7) could be observed. The total amount of impurity formed in the formulation of Table 7 after two weeks (0.69) was almost three times the starting amount (0.25), whereas the total amount of impurity formed in the formulation of Table 8 after two weeks (0.46) was only just over twice the starting amount (0.22).

After four weeks, however, the total amount of impurity formed in the formulations of Table 7 (4.63) and in the formulation of Table 8 (4.92) was very similar, as were the amounts of the main impurities, which have RRT values of 0.35, 0.60, and 1.06, respectively. In both cases, the degradation is significant, regardless of the presence or absence of lactose. Accordingly, Tables 7 and 8 show that lactose alone does not prevent the oxidation that occurs at pH values that are significantly higher than 5.5, such as 6.0 or higher.

### Comparative Example 3

The stability of formulations obtained from tigecycline in lyophilized form by dissolving it in 0.9% Normal Saline solution (a) in the absence of dextrose and (b) in the presence of dextrose was compared after storing at about −20° C. over a four-week period. No acid or base was added to control the pH. The pH values of the solutions were (a) 7.86 and (b) 7.71. The results are shown in Tables 9-10.

TABLE 9
Percentage of impurities formed over time in a 0.9% Normal Saline solution.
The impurity at RRT 0.74 corresponds to the tigecycline epimer

| | Strength, mg/ | RRT | RRT | RRT | RRT | RRT | RRT | RRT | RRT | |
|---|---|---|---|---|---|---|---|---|---|---|
| Sample | ml | 0.14 | 0.36 | 0.35 | 0.50 | 0.56 | 0.70 | 0.74 | 1.72 | Total |
| Initial | 0.51 | ND | ND | ND | ND | 0.03 | ND | 0.19 | ND | 0.22 |
| 1 week | 0.52 | ND | 0.07 | ND | ND | 0.45 | 0.08 | 0.15 | ND | 0.79 |
| 2 weeks | 0.50 | ND | 0.15 | ND | 0.05 | 0.66 | ND | 0.12 | 0.10 | 1.08 |
| 3 weeks | 0.49 | ND | 0.39 | ND | 0.09 | 1.48 | ND | 0.10 | 0.24 | 2.30 |
| 4 weeks | 0.46 | 0.75 | 0.68 | 0.08 | 0.18 | 1.20 | 0.12 | 0.11 | 0.19 | 3.31 |

ND = Not Determined

TABLE 10
Percentage of impurities formed over time in the
presence of 5% dextrose solution.

| | Strength, | RRT | RRT | RRT | RRT | RRT | RRT | |
|---|---|---|---|---|---|---|---|---|
| Sample | mg/ml | 0.19 | 0.50 | 0.56 | 0.70 | 0.74 | 1.99 | Total |
| Initial | 0.50 | ND | ND | 0.06 | ND | 0.21 | 0.15 | 0.42 |
| 1 week | 0.52 | ND | ND | 0.05 | ND | 0.34 | 0.31 | 0.75 |
| 2 weeks | 0.49 | ND | ND | 0.07 | ND | 0.49 | 0.43 | 0.99 |
| 3 weeks | 0.46 | 0.13 | ND | 0.09 | ND | 0.59 | 0.48 | 1.29 |
| 4 weeks | 0.47 | 0.13 | 0.06 | ND | ND | 1.29 | 0.29 | 1.77 |

Even in the relatively short period of four weeks, a significant increase in stability of the formulation containing dextrose (Table 11) relative to the formulation that does not contain dextrose (Table 10) could be observed. The total amount of impurity formed in the formulation of Table 10 after four weeks (3.31) was about fifteen times the starting amount (0.22), whereas the total amount of impurity formed in the formulation of Table 11 after four weeks (1.77) was slightly over four times the starting amount (0.42). The effect of dextrose is to significantly reduce the amount after four weeks of impurities having an RRT of 0.14, 0.36 and 0.56. The amount of tigecycline epimer, however, increased significantly in the presence of dextrose relative to the amount of epimer in the absence of dextrose.

### Comparative Example 4

The stability of a tigecycline vial at room temperature was analyzed over a 24 month period.

TABLE 11

| | | Percent Impurities Formed | | | | | |
|---|---|---|---|---|---|---|---|
| Sample | | RRT | RRT | | RRT | | Total impurity |
| name | Strength | 0.55 | 0.64 | Epimer | 1.25 | Minocycline | content |

| | | | | | | | |
|---|---|---|---|---|---|---|---|
| Acceptance | 96-110% | ≦0.5% | ≦0.7% | ≦3.0% | ≦0.3 | FIO | NA |
| criteria | | | | | | | |
| 9 months | 105.6 | BRL | BRL | 0.8 | 0.07 | 0.08 | 0.95 |
| 24 months | 104.9 | BRL | BRL | 1.3 | 0.09 | 0.09 | 1.48 |

RRT—Relative Retention Time
BRL—Below Reporting Level
FIO—For Information only
NA—Not Applicable

### Example 1

### Tigecycline Frozen Bags 26 Month Stability Data

A) Formulation 1: tigecycline 0.5 mg/ml, brought up to volume with 5% Dextrose, pH=about 4.7

### Formulation 1 Example

### Tigecycline 500 mg

5% Dextrose IV Solution brought up to 1000 mL
Concentration manufactured 0.5 mg/ml

### PH˜4.7

### Manufacturing Procedure:

1. Collected approximately 1000 ml of 5% dextrose injection by emptying 10 IV bags

2. Dissolved tigecycline in about 700 mL of the solution.

3. Adjusted pH to 4.72

4. Brought up to 1000 mL

5. Placed 100 mL solution in to 10 IV bags

6. Applied stoppers and seals to seal the bag.

7. Place the bags at −70° C. for stability studies

TABLE 12

| | | Percent of Degradants Formed | | | |
|---|---|---|---|---|---|
| | | | | | Total |
| | Strength, | | | | impurity |
| | mg/ml | Epimer | RRT 0.76 | Minocycline | content |
| Acceptance | NA | ≦3.0% | ≦0.2% | ≦0.19% | ≦6.0% |
| criteria | | | | | |
| 9 month | 0.49 | 0.61 | 0.09 | 0.19 | 0.89 |
| 26 month | 0.48 | 0.69 | 0.02 | 0.19 | 0.90 |

RRT—Relative Retention Time

### RRT—Relative Retention Time

As shown in the Table, even over a period of 24 months, the percentage of epimer formed was 0.69%, significantly less than the acceptance criterion for this impurity. Similar conclusions apply to the total percentage of degradants formed (0.90%, far less than the 6.0% acceptance criterion). Accordingly, the composition of Table 10, containing dextrose and at a pH between 4.0 and 5.5, shows high stability as measured both by epimer content and total impurity content.
B) Formulation 2: Tigecycline 0.5 mg/ml with Lactose Monohydrate ratio 1:2, pH=about 4.7

### Formulation 2 Example

### Tigecycline 500 mg

### Lactose Monohydrate 1000 mg

WFI (water for injection) brought up to 1000 mL
Concentration manufactured 0.5 mg/ml

### PH˜4.7

### Manufacturing Procedure:

1. Collected approximately 1000 ml WFI by emptying 10 IV bags

2. Blended tigecycline and lactose monohydrate together

3. Dissolved tigecycline in about 700 mL of WFI.

4. Adjusted pH to 4.7

5. Brought up to 1000 mL

6. Placed 100 mL solution in to 10 IV bags

7. Applied stoppers and seals to seal the bag.

8. Place the bags at −70° C. for stability studies

TABLE 13

| | | Percent of Degradants Formed | | | |
|---|---|---|---|---|---|
| | | | | | Total |
| | Strength, | | | | impurity |
| | mg/ml | Epimer | RRT 0.76 | Minocycline | content |
| Acceptance | NA | ≦3.0% | ≦0.2% | ≦0.19% | ≦6.0% |
| criteria | | | | | |
| 9 month | 0.49 | 0.44 | 0.08 | 0.18 | 0.70 |
| 26 month | 0.49 | 0.40 | 0.01 | 0.16 | 0.57 |

RRT—Relative Retention Time

### RRT—Relative Retention Time

As shown in the Table, even over a period of 24 months, the percentage of epimer formed was about 0.40%, significantly less than the acceptance criterion for this impurity and even lower than the corresponding amount for Formulation 1 in Table 12. Similar conclusions apply to the total percentage of degradants formed (0.57%, far less than the 6.0% acceptance criterion). According, the composition of Table 12, containing lactose and at a pH between 4.0 and 5.5, shows exceptionally high stability as measured both by epimer content and total impurity content.

The above shows that a formulation comprising tigecycline and_lactose may have a total impurity content equal to or less than about 0.7% after up to 26 months, and that a formulation comprising tigecycline and dextrose may have a total impurity content equal to or less than about 0.9% after up to 26 months.

### Examples 2-7

### Tigecycline Frozen Bags 6 Months Stability Data at −20° C.

In each of the examples below, the stability of a frozen bag containing tigecycline and, respectively for examples 2-8, dextrose, sucrose, glucose, ribose, mannose, and xylose, was analyzed at set time intervals of 1, 2, 3 and 6 months.

In each case the pH ranged from about 4.6 to about 4.8 and the ratio of tigecycline to dextrose, sucrose, glucose, ribose, mannose, or xylose was 1:2.
WFI was brought up to 1000 mL in each case.

### Example 2

### Tigecycline/Dextrose

| | Initial | | 1 month | | 2 months | | 3 months | | 6 months | |
|---|---|---|---|---|---|---|---|---|---|---|
| Concentration | 0.5/1 | 1.5/3 | 0.5/1 | 1.5/3 | 0.5/1 | 1.5/3 | 0.5/1 | 1.5/3 | 0.5/1 | 1.5/3 |
| Tigecycline/Dextrose, | | | | | | | | | | |
| Strength of | 0.50 | 1.50 | 0.49 | 1.52 | 0.50 | 1.51 | 0.48 | 1.47 | 0.48 | 1.40 |
| Tigecycline, | | | | | | | | | | |
| mg/ml | | | | | | | | | | |
| Epimer, % | 0.21 | 0.23 | 1.50 | 1.54 | 2.0 | 1.81 | 3.20 | 3.1 | 5.13 | 5.34 |
| Ph | 4.69 | 4.72 | 4.67 | 4.70 | 4.72 | 4.65 | 4.72 | 4.73 | 4.47 | 4.44 |
| Particulate count | | | | | | | | | | |
| 25 | NA | NA | 176 | 133 | 188 | 158 | 100 | 171 | 82 | 155 |
| 10 | | | 9 | 8 | 13 | 10 | 3 | 5 | 8 | 8 |

### Example 3

### Tigecycline/Sucrose

| | Initial | | 1 month | | 2 months | | 3 months | | 6 months | |
|---|---|---|---|---|---|---|---|---|---|---|
| Concentration | 0.5/1 | 1.5/3 | 0.5/1 | 1.5/3 | 0.5/1 | 1.5/3 | 0.5/1 | 1.5/3 | 0.5/1 | 1.5/3 |
| Tigecycline/Sucrose | | | | | | | | | | |
| Strength of | 0.50 | 1.50 | 0.50 | 1.51 | 0.50 | 1.50 | 0.49 | 1.48 | 0.48 | 1.42 |
| Tigecycline, | | | | | | | | | | |
| mg/ml | | | | | | | | | | |
| Epimer, % | 0.20 | 0.22 | 0.42 | 0.50 | 0.84 | 0.65 | 1.40 | 1.15 | 3.16 | 2.69 |
| pH | 4.68 | 4.72 | 4.63 | 4.76 | 4.69 | 4.72 | 4.81 | 4.93 | 4.53 | 4.79 |
| Particulate count | | | | | | | | | | |
| 25 | NA | NA | 85 | 153 | 99 | 169 | 121 | 164 | 135 | 215 |
| 10 | | | 4 | 9 | 5 | 7 | 6 | 7 | 4 | 8 |

### Example 4

### Tigecycline/Glucose

| | Initial | | 1 month | | 2 months | | 3 months | | 6 months | |
|---|---|---|---|---|---|---|---|---|---|---|
| Concentration | 0.5/1 | 1.5/3 | 0.5/1 | 1.5/3 | 0.5/1 | 1.5/3 | 0.5/1 | 1.5/3 | 0.5/1 | 1.5/3 |
| Tigecycline/Glucose | | | | | | | | | | |
| Strength of | 0.50 | 1.50 | 0.50 | 1.50 | 0.50 | 1.50 | 0.48 | 1.45 | 0.48 | 1.41 |
| Tigecycline, | | | | | | | | | | |
| mg/ml | | | | | | | | | | |
| Epimer, % | 0.15 | 0.20 | 1.25 | 1.20 | 1.89 | 1.60 | 3.04 | 3.05 | 5.88 | 5.50 |
| Other Impurities, % | ND | ND | ND | ND | ND | 0.10 | ND | ND | 0.07 | 0.06 |
| | | | | | | (RRT 0.60) | | | (RRT 0.60 | (RRT 0.60 |
| pH | 4.78 | 4.70 | 4.81 | 4.63 | 4.75 | 4.68 | 4.94 | 4.76 | 4.65 | 4.36 |
| Particulate count | | | | | | | | | | |
| 25 | NA | NA | 201 | 188 | 187 | 169 | 83 | 116 | 122 | 171 |
| 10 | | | 16 | 11 | 9 | 13 | 5 | 8 | 2 | 4 |

### Example 5

### Tigecycline/Ribose

| | Initial | | 1 month | | 2 month | | 3 months | | 6 months | |
|---|---|---|---|---|---|---|---|---|---|---|
| Concentration | 0.5/1 | 1.5/3 | 0.5/1 | 1.5/3 | 0.5/1 | 1.5/3 | 0.5/1 | 1.5/3 | 0.5/1 | 1.5/3 |
| of | | | | | | | | | | |
| Tigecycline/Ribose | | | | | | | | | | |
| Strength of | 0.50 | 1.50 | 0.50 | 1.50 | 0.50 | 1.50 | 0.48 | 1.45 | 0.48 | 1.40 |
| Tigecycline, | | | | | | | | | | |
| mg/ml | | | | | | | | | | |
| Epimer, % | 0.15 | 0.19 | 1.47 | 1.43 | 1.87 | 1.68 | 3.02 | 3.17 | 5.71 | 5.77 |
| Other impurities, % | ND | ND | ND | ND | ND | 0.09 | 0.06 | 0.06 | 0.09 | 0.10 |
| | | | | | | (RRT 0.60) | (RRT 0.60) | (RRT 0.60) | (RRT 0.60) | (RRT 0.60) |
| pH | 4.71 | 4.66 | 4.68 | 4.56 | 4.72 | 4.63 | 4.82 | 4.68 | 4.47 | 4.34 |
| Particulate count | | | | | | | | | | |
| 25 | NA | NA | 138 | 221 | 152 | 236 | 169 | 217 | 165 | 201 |
| 10 | | | 11 | 22 | 10 | 17 | 8 | 25 | 1 | 3 |

### Example 6

### Tigecycline/Mannose

| | Initial | | 1 month | | 2 months | | 3 months | | 6 months | |
|---|---|---|---|---|---|---|---|---|---|---|
| Concentration of | 0.5/1 | 1.5/3 | 0.5/1 | 1.5/3 | 0.5/1 | 1.5/3 | 0.5/1 | 1.5/3 | 0.5/1 | 1.5/3 |
| Tigecycline/Mannose | | | | | | | | | | |
| Strength of | 0.50 | 1.51 | 0.50 | 1.50 | 0.50 | 1.50 | 0.49 | 1.45 | 0.50 | 1.44 |
| Tigecycline, | | | | | | | | | | |
| mg/ml | | | | | | | | | | |
| Epimer, % | 0.16 | 0.19 | 0.89 | 0.62 | 1.25 | 1.12 | 1.92 | 2.31 | 3.69 | 4.0 |
| pH | 4.73 | 4.68 | 4.79 | 4.64 | 4.83 | 4.59 | 4.99 | 4.81 | 4.54 | 4.44 |
| Particulate count | | | | | | | | | | |
| 25 | NA | NA | 243 | 409 | 235 | 398 | 273 | 389 | 333 | 415 |
| 10 | | | 18 | 24 | 13 | 18 | 13 | 10 | 3 | 1 |

### Example 7

### Tigecycline/Xylose

| | Initial | | 1 month | | 2 months | | 3 months | | 6 months | |
|---|---|---|---|---|---|---|---|---|---|---|
| Concentration of | 0.5/1.0 | 1.5/3 | 0.5/1.0 | 1.5/3 | 0.5/1.0 | 1.5/3 | 0.5/1.0 | 1.5/3 | 0.5/1.0 | 1.5/3 |
| Tigecycline/Xylose | | | | | | | | | | |
| Strength of | 0.50 | 1.48 | 0.50 | 1.49 | 0.50 | 1.48 | 0.49 | 1.46 | 0.47 | 1.30 |
| Tigecycline, | | | | | | | | | | |
| mg/ml | | | | | | | | | | |
| Epimer, % | 0.16 | 0.15 | 0.34 | 0.27 | 0.89 | 1.62 | 2.02 | 1.68 | 4.68 | 3.88 |
| Other Impurities % | ND | ND | ND | ND | ND | ND | ND | ND | 0.06 | 0.08 |
| | | | | | | | | | (RRT 0.50) | (RRT 0.50) |
| | | | | | | | | | 0.10 | 0.17 |
| | | | | | | | | | (RRT 0.60) | (RRT 0.60) |
| Ph | 4.82 | 4.72 | 4.89 | 4.83 | 4.70 | 4.94 | 4.76 | 4.81 | 4.97 | 4.70 |
| Particulate count | | | | | | | | | | |
| 25 | NA | 303 | 168 | 298 | 180 | 329 | 151 | 108 | 228 | 105 |
| 10 | | 18 | 11 | 12 | 9 | 16 | 8 | 4 | 5 | 3 |

As shown in the table for each of Examples 2-7, after a 2-month period none of the compositions showed a percentage of epimer formed greater than 2.0%, significantly less than the 3% acceptance criterion for this impurity, indicating that all of the above formulations would remain viable after at least a 2-month storage period.

Without wishing to be bound by theory or mechanism, the stabilization of epimer formation by agents such as lactose is believed to be due to a favorable spatial orientation of the agent such as lactose and tigecycline molecules that may result from ion-pair interactions, hydrophobic interactions, and/or hydrogen bonding.

While particular embodiments of the invention have been illustrated, they are not limiting of the invention, and one skilled in the art will readily envision changes and modifications that can be made without departing from the scope of the invention, which is defined by the claims appended herein.

We claim:

1. A frozen pharmaceutical formulation suitable for administration to a subject parenterally, comprising a therapeutically effective amount of tigecycline and an agent selected from the group consisting of lactose, dextrose, glucose, mannose, sucrose, ribose, xylose and a combination thereof, wherein the formulation in a pre-frozen state at about 22° C. or in an unfrozen state at about 22° C. has a pH in the range of from 4.0 to 5.5.

2. The formulation of claim 1, wherein the agent is selected from the group consisting of lactose, dextrose, glucose, mannose, and a combination thereof.

3. The formulation of claim 1, wherein the concentration of tigecycline epimer in the formulation is at or below about 3% of the concentration of tigecycline.

4. The formulation of claim 3, wherein the agent is lactose.

5. The formulation of claim 1, wherein the therapeutically effective amount of tigecycline is about 150 mg.

6. The formulation of claim 1, wherein tigecycline is in a concentration within the range of about 0.1 mg/ml to about 2.0 mg/ml.

7. The formulation of claim 6, wherein tigecycline is in a concentration within the range of about 0.5 to about 1.5 mg/ml.

8. The formulation of claim 1, wherein the pH is from 4.5 to 5.1.

9. The formulation of claim 1, wherein the pH is from 4.8 to 5.1.

10. The formulation of claim 1, wherein the agent is lactose.

11. The formulation of claim 10, wherein lactose is in the form of lactose monohydrate.

12. The formulation of claim 10, wherein lactose is in a concentration within the range of about 40 mg/ml to about 80 mg/ml.

13. The formulation of claim 1, wherein the formulation contains at least about 98.0% tigecycline as a percentage of the initial weight of tigecycline in the composition after up to about 42 days at a temperature of about −20° C.

14. The formulation of claim 1, wherein the formulation contains at least about 90.0% tigecycline as a percentage of the initial weight of tigecycline in the composition after up to 36 months at a temperature of about −20° C. or lower than −20° C.

15. The formulation of claim 14, wherein the temperature is of about −20° C. to about −70° C.

16. The formulation of claim 15, wherein the formulation contains at least about 90.0% tigecycline as a percentage of the initial weight of tigecycline in the composition after up to 6 months.

17. The formulation of claim 13, wherein the pH is about 5.0.

18. The formulation of claim 1, wherein the agent is lactose and the formulation has a total impurity content equal to or less than about 0.7% after up to 26 months.

19. The formulation of claim 1, wherein the agent is dextrose and the formulation has a total impurity content equal to or less than about 0.9% after up to 26 months.

20. A process of making a frozen pharmaceutical formulation, comprising the steps of:
a. dissolving tigecycline and an agent selected from the group consisting of lactose, dextrose, glucose, mannose, sucrose, ribose, xylose and a combination thereof into a suitable liquid, forming a premixed solution;
b. adjusting the pH of the premixed solution to a range of 4.0-5.5;
c. filling one or more containers with the premixed solution; and
d. storing the one or more containers of premixed solution at a temperature below the freezing point of the liquid.

21. The process of claim 20, wherein the suitable liquid is water.

22. The process of claim 21, wherein the temperature below the freezing point of the liquid is about −20° C.

23. The process of claim 21, wherein the temperature below the freezing point of the liquid is about −20° C. to about −70° C.

24. The process of claim 20, wherein the pH of the premixed solution is from 4.5 to 5.1.

25. A pharmaceutical formulation made by the process of claim 20.

26. A process of making a frozen pharmaceutical formulation, comprising the steps of:
a. dissolving tigecycline and an agent selected from the group consisting of lactose, dextrose, glucose, mannose sucrose, fructose, ribose, xylose and a combination thereof into a suitable liquid having a freezing point above about −20° C., forming a premixed solution;
b. filling one or more containers with the premixed solution;
c. storing the one or more containers of premixed solution at a temperature below the freezing point of the liquid; and
d. adding an acidifying agent to form a mixture wherein the amount of acidifying agent is such that the pH of the resulting mixture when measured at a temperature of about 22° C. is 4.0-5.5.

27. The process of claim 25, wherein the suitable liquid is water.

28. The process of claim 26, wherein the temperature below the freezing point of the liquid is about −20° C.

29. The process of claim 26, wherein the temperature below the freezing point of the liquid is about −20° C. to about −70° C.

30. The process of claim 27, wherein the pH of the premixed solution is from 4.5 to 5.1.

31. A pharmaceutical formulation made by the process of claim 26.

32. A kit comprising (a) a flexible bag, and (b) the frozen pharmaceutical formulation of claim 1, wherein the frozen pharmaceutical formulation is contained in the bag.

33. A kit comprising (a) a vial, and (b) the frozen pharmaceutical formulation of claim 1, wherein the frozen pharmaceutical formulation is contained in the vial.
